(19) United States
(12) Patent Application Publication
(10) Pub. No.: US 2023/0002453 A1
(43) Pub. Date: Jan. 5, 2023

(54) GENE EDITING SYSTEM DERIVED FROM FLAVOBACTERIA

(71) Applicant: Shanghai Bluecross Medical Science Institute, Shanghai (CN)

(72) Inventors: Caixia Gao, Beijing (CN); Shuai Jin, Beijing (CN)

(73) Assignee: Shanghai Bluecross Medical Science Institute, Shanghai (CN)

(21) Appl. No.: 17/777,936

(22) PCT Filed: Nov. 18, 2020
(86) PCT No.: PCT/CN2020/129665

§ 371 Date: May 18, 2022

(30) Foreign Application Priority Data
Nov. 18, 2019 (CN) 201911126348.4

(51) Int. Cl. C07K 14/195; C12N 15/90; C12N 5/10

(52) U.S. Cl. CPC C07K 14/195; C12N 15/90; C12N 5/10

(57) ABSTRACT

The present invention belongs to the field of genetic engineering. Specifically, the present invention relates to a gene editing system derived from Flavobacterium and uses thereof.

### CROSS REFERENCE TO RELATED APPLICATIONS

This application is a U.S. National Stage application of PCT International Patent Application Serial No. PCT/CN2020/129665, filed Nov. 18, 2020, which itself claims the benefit of Chinese Patent Application Serial No. 201911126348.4, filed Nov. 18, 2019, the disclosure of each of which incorporated herein by reference in its entirety.

### REFERENCE TO SEQUENCE LISTING SUBMITTED ELECTRONICALLY

The content of the electronically submitted sequence listing in ASCII text file (Name: 1547_42_PCT_US_ST25.txt; Size: 45 kilobytes; and Date of Creation: May 18, 2022) filed with the instant application is incorporated herein by reference in its entirety.

### TECHNICAL FIELD

The present invention belongs to the field of genetic engineering. Specifically, the present invention relates to a gene editing system derived from Flavobacterium and uses thereof.

### BACKGROUND

Genome editing technology is a genetic engineering technology based on the targeted modification of the genome by artificial nucleases, and is playing an increasingly powerful role in agricultural and medical research. Clustered regularly interspaced short palindromic repeats/CRISPR associated (CRISPR) systems are currently the most widely used genome editing tools. Proteins can target any location in the genome, resulting in a double-strand break (DSB) at the targeted sequence, activating intracellular Non-homologous End Joining (NHEJ) or homologous repair (Homology Directly Repair, HDR) pathways to introduce mutations. The most commonly used Cas protein is the Cas9 protein derived from Streptococcus pyogenes, which belongs to the Type II-A subtype in the Class II CRISPR system. Cong et al. (Multiplex Genome Engineering Using CRISPR/Cas Systems, Science, 2013) and Mali et al. (RNA-guided human genome engineering via Cas9, Science, 2013) successfully applied the CRISPR/Cas9 system in human cell lines.

Both the CRISPR/Cas12a system and the CRISPR/Cas9 system belong to the Class II CRISPR system. Zetsche et al. applied the Cas12a protein (formerly Cpf1) derived from Streptococcus aminoacids and Lachnospira for the first time to gene editing of animal cells (Cpf1 is a Single RNA-Guided Endonuclease of a Class 2 CRISPR-Cas System, Cell, 2015). The difference is that the CRISPR/Cas12a system belongs to the Type V type, which has a shorter crRNA sequence and higher specificity. The advantages further expand the gene editing toolbox of the CRISPR system.

So far, gene editing tools based on CRISPR/Cas9 and CRISPR/Cas12 have been successfully used in animal cell lines, animal individuals, plant cells, plant individuals and microorganisms. The tools have caused a revolution in the field of gene editing. However, the working efficiency of the CRISPR/Cas12a system varies greatly at different target sites, and the working efficiency is lower at some sites in the plant genome, which may be due to the fact that the existing Cas12a systems are mainly derived from pathogenic bacteria of human or animal and the optimal working temperature is higher than that in plants. Therefore, it is necessary to identify and develop a CRISPR/Cas12a system that can work stably at suitable plant temperatures.

### SUMMARY

The inventors found the unreported FbCas12a protein in plant symbiotic bacteria through homologous similarity comparison, and artificially predicted the mature form of its own crRNA, and compared its own crRNA with the crRNA of LbCas12a in vivo efficiency. It was found that FbCas12a can work in plant cells and has higher editing efficiency when using the crRNA of LbCas12a.

### BRIEF DESCRIPTION OF FIGURES

FIG. 1. Alignment results of DR sequences and protein sequences of Cas12a from different sources.

FIG. 2. Schematic diagram of the carrier used in the examples.

FIG. 3. Editing of the rice endogenous gene OsEPSPS by the combination of FbCas12a and FbcrRNA.

FIG. 4. The result of the combination of FbCas12a and FbcrRNA or LbcrRNA on rice endogenous gene editing.

### DETAILED DESCRIPTION

### 1. Definitions

In the present invention, unless indicated otherwise, the scientific and technological terminologies used herein refer to meanings commonly understood by a person skilled in the art. Also, the terminologies and experimental procedures used herein relating to protein and nucleotide chemistry, molecular biology, cell and tissue cultivation, microbiology, immunology, all belong to terminologies and conventional methods generally used in the art. For example, the standard DNA recombination and molecular cloning technology used herein are well known to a person skilled in the art, and are described in details in the following references: Sambrook, J., Fritsch, E. F. and Maniatis, T., Molecular Cloning: A Laboratory Manual; Cold Spring Harbor Laboratory Press: Cold Spring Harbor, 1989. In the meantime, in order to better understand the present invention, definitions and explanations for the relevant terminologies are provided below.

As used herein, the term “and/or” encompasses all combinations of items connected by the term, and each combination should be regarded as individually listed herein. For example, “A and/or B” covers “A”, “A and B”, and “B”. For example, “A, B, and/or C” covers “A”, “B”, “C”, “A and B”, “A and C”, “B and C”, and “A and B and C”.

When the term “comprise” is used herein to describe the sequence of a protein or nucleic acid, the protein or nucleic acid may consist of the sequence, or may have additional amino acids or nucleotide at one or both ends of the protein or nucleic acid, but still have the activity described in this invention. In addition, those skilled in the art know that the methionine encoded by the start codon at the N-terminus of the polypeptide will be retained under certain practical conditions (for example, when expressed in a specific expression system), but does not substantially affect the function of the polypeptide. Therefore, when describing the amino acid sequence of specific polypeptide in the specification and claims of the present application, although it may not include the methionine encoded by the start codon at the N-terminus, the sequence containing the methionine is also encompassed, correspondingly, its coding nucleotide sequence may also contain a start codon; vice versa.

“Genome” as used herein encompasses not only chromosomal DNA present in the nucleus, but also organelle DNA present in the subcellular components (e.g., mitochondria, plastids) of the cell.

As used herein, “organism” includes any organism that is suitable for genome editing, eukaryotes are preferred. Examples of organisms include, but are not limited to, mammals such as humans, mice, rats, monkeys, dogs, pigs, sheep, cattle, cats; poultry such as chickens, ducks, geese; plants including monocots and dicots such as rice, corn, wheat, sorghum, barley, soybean, peanut, Arabidopsis and the like.

A “genetically modified organism” or “genetically modified cell” includes the organism or the cell which comprises within its genome an exogenous polynucleotide or a modified gene or expression regulatory sequence. For example, the exogenous polynucleotide is stably integrated within the genome of the organism or the cell such that the polynucleotide is passed on to successive generations. The exogenous polynucleotide may be integrated into the genome alone or as part of a recombinant DNA construct. The modified gene or expression regulatory sequence means that, in the organism genome or the cell genome, said sequence comprises one or more nucleotide substitution, deletion, or addition.

The term “exogenous” with respect to sequence means a sequence that originates from a foreign species, or, if from the same species, is substantially modified from its native form in composition and/or genomic locus by deliberate human intervention.

“Polynucleotide”, “nucleic acid sequence”, “nucleotide sequence”, or “nucleic acid fragment” are used interchangeably to refer to a polymer of RNA or DNA that is single- or double-stranded, optionally containing synthetic, non-natural or altered nucleotide bases. Nucleotides (usually found in their 5′-monophosphate form) are referred to by their single letter designation as follows: “A” for adenylate or deoxyadenylate (for RNA or DNA, respectively), “C” for cytidylate or deoxycytidylate, “G” for guanylate or deoxyguanylate, “U” for uridylate, “T” for deoxythymidylate, “R” for purines (A or G), “Y” for pyrimidines (C or T), “K” for G or T, “H” for A or C or T, “I” for inosine, and “N” for any nucleotide.

“Polypeptide”, “peptide”, “amino acid sequence” and “protein” are used interchangeably herein to refer to a polymer of amino acid residues. The terms apply to amino acid polymers in which one or more amino acid residue is an artificial chemical analogue of a corresponding naturally occurring amino acid, as well as to naturally occurring amino acid polymers. The terms “polypeptide”, “peptide”, “amino acid sequence”, and “protein” are also inclusive of modifications including, but not limited to, glycosylation, lipid attachment, sulfation, gamma-carboxylation of glutamic acid residues, hydroxylation and ADP-ribosylation.

Sequence “identity” has recognized meaning in the art, and the percentage of sequence identity between two nucleic acids or polypeptide molecules or regions can be calculated using the disclosed techniques. Sequence identity can be measured along the entire length of a polynucleotide or polypeptide or along a region of the molecule. (See, for example, Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991). Although there are many methods for measuring the identity between two polynucleotides or polypeptides, the term “identity” is well known to the skilled person (Carrillo, H. & Lipman, D., SIAM J Applied Math 48: 1073 (1988)).

Suitable conserved amino acid replacements in peptides or proteins are known to those skilled in the art and can generally be carried out without altering the biological activity of the resulting molecule. In general, one skilled in the art recognizes that a single amino acid replacement in a non-essential region of a polypeptide does not substantially alter biological activity (See, for example, Watson et al., Molecular Biology of the Gene, 4th Edition, 1987, The Benjamin/Cummings Pub. co., p. 224).

As used herein, an “expression construct” refers to a vector suitable for expression of a nucleotide sequence of interest in an organism, such as a recombinant vector. “Expression” refers to the production of a functional product. For example, the expression of a nucleotide sequence may refer to transcription of the nucleotide sequence (such as transcribe to produce an mRNA or a functional RNA) and/or translation of RNA into a protein precursor or a mature protein.

“Expression construct” of the invention may be a linear nucleic acid fragment, a circular plasmid, a viral vector, or, in some embodiments, an RNA that can be translated (such as an mRNA).

“Expression construct” of the invention may comprise regulatory sequences and nucleotide sequences of interest that are derived from different sources, or regulatory sequences and nucleotide sequences of interest derived from the same source, but arranged in a manner different than that normally found in nature.

“Regulatory sequence” or “regulatory element” are used interchangeably and refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences may include, but are not limited to, promoters, translation leader sequences, introns, and polyadenylation recognition sequences.

“Promoter” refers to a nucleic acid fragment capable of controlling the transcription of another nucleic acid fragment. In some embodiments of the present invention, the promoter is a promoter capable of controlling the transcription of a gene in a cell, whether or not it is derived from the cell. The promoter may be a constitutive promoter or a tissue-specific promoter or a developmentally-regulated promoter or an inducible promoter.

“Constitutive promoter” refers to a promoter that may cause expression of a gene in most circumstances in most cell types. “Tissue-specific promoter” and “tissue-preferred promoter” are used interchangeably, and refer to a promoter that is expressed predominantly but not necessarily exclusively in one tissue or organ, but that may also be expressed in one specific cell or cell type. “Developmentally regulated promoter” refers to a promoter whose activity is determined by developmental events. “Inducible promoter” selectively expresses a DNA sequence operably linked to it in response to an endogenous or exogenous stimulus (environment, hormones, or chemical signals, and so on).

As used herein, the term “operably linked” means that a regulatory element (for example but not limited to, a promoter sequence, a transcription termination sequence, and so on) is associated to a nucleic acid sequence (such as a coding sequence or an open reading frame), such that the transcription of the nucleotide sequence is controlled and regulated by the transcriptional regulatory element. Techniques for operably linking a regulatory element region to a nucleic acid molecule are known in the art.

“Introduction” of a nucleic acid molecule (e.g., plasmid, linear nucleic acid fragment, RNA, etc.) or protein into an organism means that the nucleic acid or protein is used to transform a cell of the organism such that the nucleic acid or protein functions in the cell. As used in the present invention, “transformation” includes both stable and transient transformations.

“Stable transformation” refers to the introduction of an exogenous nucleotide sequence into the genome, resulting in the stable inheritance of foreign genes. Once stably transformed, the exogenous nucleic acid sequence is stably integrated into the genome of the organism and any of its successive generations.

“Transient transformation” refers to the introduction of a nucleic acid molecule or protein into a cell, performing its function without the stable inheritance of an exogenous gene. In transient transformation, the exogenous nucleic acid sequence is not integrated into the genome.

“Trait” refers to the physiological, morphological, biochemical, or physical characteristics of a cell or an organism.

“Agronomic trait” is a measurable parameter including but not limited to, leaf greenness, yield, growth rate, biomass, fresh weight at maturation, dry weight at maturation, fruit yield, seed yield, total plant nitrogen content, fruit nitrogen content, seed nitrogen content, nitrogen content in a vegetative tissue, total plant free amino acid content, fruit free amino acid content, seed free amino acid content, free amino acid content in a vegetative tissue, total plant protein content, fruit protein content, seed protein content, protein content in a vegetative tissue, drought tolerance, nitrogen uptake, root lodging, harvest index, stalk lodging, plant height, ear height, ear length, disease resistance, cold resistance, salt tolerance, and tiller number and so on.

### 2. Genome Editing System Based on Flavobacterium Cas12a Protein

In one aspect, the present invention provides a novel Cas12a protein which comprises

(i) an amino acid sequence having at least 80%, at least 85%, at least 90%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9%, or even 100% sequence identity to SEQ ID NO: 1, or

(ii) an amino acid sequence having one or more, such as 1, 2, 3, 4, 5, 6, 7, 8, 9 or 10 amino acid substitutions, deletions or additions as compared to SEQ ID NO: 1.

“Cas12a protein”, “Cas12a nuclease” and “Cas12a” are used interchangeably herein to refer to an RNA-guided nuclease or variant thereof including the Cas12a protein or a fragment thereof. Cas12a is a component of the CRISPR-Cas12a genome editing system, which can target and/or cleave a DNA target sequence under the guidance of guide RNA (crRNA) to form a DNA double-strand break (DSB). The Cas12a protein of the present invention is derived from plant symbiotic bacteria and, therefore, is particularly suitable for genome editing in plants.

In some embodiments of various aspects herein, the Cas12a protein is derived from a species from Flavobacterium. In some embodiments, the Cas12a protein is derived from Flavobacterium branchiophilum. Those skilled in the art will understand that Cas12a proteins in different strains of a same bacterial species may have certain differences in amino acid sequence, but can achieve substantially the same function.

In some embodiments of various aspects of the invention, the Cas12a protein is recombinantly produced. In some embodiments of various aspects of the invention, the Cas12a protein further contains a fusion tag, e.g., a tag used for Cas12a protein isolation/or purification. Methods for recombinant production of proteins are known in the art. A variety of tags that can be used to isolate and/or purify proteins are known in the art, including but not limited to His tags, GST tags, and the like. Generally, these tags do not alter the activity of the protein of interest. In some embodiments, the Cas12a protein is also fused with other functional proteins, such as deaminase, transcriptional activator/repressor protein, etc., so as to realize base editing or transcriptional regulation functions.

In some embodiments of various aspects of the invention, the Cas12a proteins of the invention further comprise a nuclear localization sequence (NLS), e.g., is linked to a nuclear localization sequence through a linker. The a “linker” may be a non-functional amino acid sequence which is 1-50 (e.g., 1, 2, 3, 4, 5, 6, 7, 8, 9, 10, 11, 12, 13, 14, 15, 16, 17, 18, 19, 20 or 20-25 or 25-50) or more amino acids in length and is free of secondary or higher structures. For example, the linker can be a flexible linker such as SGGS (SEQ ID NO:3). In general, one or more NLSs in the Cas12a protein should be of sufficient strength to drive the accumulation of the Cas12a protein in the nucleus in an amount that enables its genome editing function. In general, the strength of nuclear localization activity is determined by the number, location of NLSs in the Cas12a protein, one or more specific NLSs used, or a combination of these factors. Exemplary nuclear localization sequences include, but are not limited to, the SV40 nuclear localization signal sequence (e.g., set forth in SEQ ID NO:4), the nucleoplasmin nuclear localization signal sequence (e.g., set forth in SEQ ID NO:5). In addition, according to the DNA position to be edited, the Cas12a protein of the present invention may also include other localization sequences, such as cytoplasmic localization sequences, chloroplast localization sequences, mitochondrial localization sequences, and the like. In some embodiments, the plurality of localization sequences can be linked by linkers. In some specific embodiments, the Cas12a protein comprises the amino acid sequence shown in SEQ ID NO:6.

In one aspect, the present invention provides the use of the Cas12a protein of the present invention for genome editing in a cell, preferably a eukaryotic cell, more preferably a plant cell.

In one aspect, the present invention provides a genome editing system for site-directed modification of a target nucleic acid sequence in the genome of a cell, comprising the Cas12a protein of the present invention and/or an expression construct comprising a nucleotide sequence encoding the Cas12a protein of the present invention.

As used herein, the “genome editing system” and “gene editing system” are interchangeable used and refers to a combination of components required for genome editing of the genome in a cell. The individual components, e.g., the Cas12a protein, the gRNA, or the corresponding expression construct, etc., of the system may be present independently of each other, or may be present in any combination as a composition.

In some embodiments, the genome editing system further comprises at least one guide RNA (gRNA) and/or an expression construct comprising a nucleotide sequence encoding the at least one guide RNA.

“Guide RNA” and “gRNA” are used interchangeably herein. The guide RNA of the CRISPR-Cas12a genome editing system is usually composed of only a crRNA molecule, wherein the crRNA contains sufficient identity to the target sequence to hybridize with the complement sequence of the target sequence and guides the CRISPR complex (Cas12a+crRNA) to bind with the target sequence in a sequence specific manner.

In some embodiments of the methods of the invention, the guide RNA is a crRNA. In some embodiments, the guide RNA comprises the crRNA scaffold sequence set forth in SEQ ID NO: 10 or 11. In some preferred embodiments, the crRNA scaffold sequence is SEQ ID NO:11. In some embodiments, the cRNA sequence further includes a sequence (i.e., a spacer sequence) located at 3′ of the cRNA scaffold sequence that specifically hybridizes to the complement of the target sequence.

In some embodiments, the crRNA comprises the following sequence:

i) 5′-AAUUUCUACUAUUGUAGAU (SEQ ID NO: 10)-Nx-3′; or

ii) 5′-UAAUUUCUACUAAGUGUAGAU (SEQ ID NO: 11)-Nx-3′;

wherein Nx represents a nucleotide sequence consisting of X consecutive nucleotides, and N is independently selected from A, G, C and U; X is an integer of 18≤X≤35, preferably, X=20, 21, 22 or 23. In some embodiments, the sequence Nx (spacer sequence) is capable of specifically hybridizing to the complement of the target sequence.

In general, a protospacer adjacent motif (PAM) is needed at the 5′ of the target sequence to be targeted by the genome editing system of the present invention. The PAM may be, for example, 5′-TTTN, where N represents A, G, C or T. However, different PAM sequences can also be used. Based on the presence of PAMs, those skilled in the art can readily determine target sequences in the genome that can be used for targeting and optionally editing and design suitable guide RNAs accordingly. For example, if there is a PAM sequence 5′-TTTG-3′ in the genome, about 18 to about 35, preferably 20, 21, 22 or 23 consecutive nucleotides immediately adjacent to its 3′ can be used as the target sequence.

In some embodiments, the at least one guide RNA is encoded by different expression constructs. In some embodiments, the at least one guide RNA is encoded by a same expression construct. In some embodiments, the at least one guide RNA and the Cas12a protein of the invention are encoded by the same expression construct.

For example, in some embodiments, the genome editing system may comprise any one selected from the group consisting of:

i) the Cas12a protein of the present invention and the at least one guide RNA, optionally, the Cas12a protein and the at least one guide RNA form a complex;

ii) an expression construct comprising a nucleotide sequence encoding the Cas12a protein of the invention, and the at least one guide RNA;

iii) a Cas12a protein of the present invention, and an expression construct comprising a nucleotide sequence encoding the at least one guide RNA;

iv) an expression construct comprising a nucleotide sequence encoding the Cas12a protein of the invention, and an expression construct comprising a nucleotide sequence encoding the at least one guide RNA;

v) an expression construct comprising a nucleotide sequence encoding the Cas12a protein of the invention and a nucleotide sequence encoding the at least one guide RNA.

In order to obtain efficient expression in a cell, in some embodiments of the present invention, the nucleotide sequence encoding the Cas12a protein is codon-optimized for the organism from which the cell to be genome edited is derived.

Codon optimization refers to a method for replacing at least one codon in the natural sequence (for example, about or more than about 1, 2, 3, 4, 5, 10, 15, 20, 25, 50 or more codons) with a codon used more frequently or most frequently in the gene of the host cell, and maintaining the natural amino acid sequence while modifying the nucleic acid sequence to enhance expression in the host cell of interest. Different species exhibit specific preferences for certain codons of specific amino acids. Codon preference (difference in codon usage between organisms) is often related to the translation efficiency of messenger RNA (mRNA), which is considered as depending on the nature of the codon being translated and the availability of the specific transfer RNA (tRNA) molecule. The advantages of the selected tRNA in the cell generally reflect the codons most frequently used for peptide synthesis. Therefore, genes may be tailored to the optimal gene expression in a given organism based on codons optimization. The codon usage tables may be easily obtained, for example, in the codon usage database (“Codon Usage Database”) available at www.kazusa.orjp/codon/, and these tables may be adjusted and applied in different ways. See Nakamura Y. et al., “Codon usage tabulated from the international DNA sequence databases: status for the year 2000”. Nucl. Acids Res., 28: 292 (2000).

The organism from which the cell that can be genome-edited by the Cas12a protein or the genome editing system of the present invention is derived is preferably an eukaryote, including but not limited to, a mammal such as human, mouse, rat, monkey, dog, pig, sheep, cattle, cat; poultry such as chicken, duck, goose; a plant including monocotyledonous and dicotyledonous plant, such as rice, corn, wheat, sorghum, barley, soybean, peanut, Arabidopsis, etc. Preferably, the Cas12a protein or genome editing system of the present invention is particularly suitable for genome editing in plants due to being derived from plant symbiotic bacteria.

In some embodiments of the present invention, the nucleotide sequence encoding the Cas12a protein is codon-optimized for plants such as rice.

In some specific embodiments, the nucleotide sequence encoding the Cas12a protein is selected from the group consisting of SEQ ID NO:2 and SEQ ID NO:7.

In some embodiments of the invention, the nucleotide sequence encoding the Cas12a protein and/or the nucleotide sequence encoding the at least one guide RNA is operably linked to an expression regulatory element, such as a promoter.

Examples of promoters that can be used in the present invention include but are not limited to polymerase (pol) I, pol II or pol III promoters. Examples of pol I promoters include chicken RNA pol I promoter. Examples of pol II promoters include but are not limited to cytomegalovirus immediate early (CMV) promoter, rous sarcoma virus long terminal repeat (RSV-LTR) promoter and simian virus 40 (SV40) immediate early promoter. Examples of pol III promoters include U6 and H1 promoter. Inducible promoter such as metalothionein promoter can be used. Other examples of promoters include T7 bacteriophage promoter, T3 bacteriophage promoter, β-galactosidase promoter and Sp6 bacteriophage promoter etc. When used for plants, promoters that can be used include but are not limited to cauliflower mosaic virus 35S promoter, maize Ubi-1 promoter, wheat U6 promoter, rice U3 promoter, maize U3 promoter and rice actin promoter etc.

In some embodiments, for precise production of guide RNA within the cell, in the expression construct comprising a nucleotide sequence encoding the at least one guide RNA, the 5′ end of the guide RNA coding sequence is linked to 3′ end of a first ribozyme coding sequence, wherein the first ribozyme is designed to cleave the first ribozyme-guide RNA fusion RNA transcribed in the cell at the 5′ end of the guide RNA, thereby forming a guide RNA that does not carry additional nucleotide at the 5′ end. In one embodiment, the 3′ end of the guide RNA coding sequence is linked to the 5′ end of a second ribozyme coding sequence, wherein the second ribozyme is designed to cleave the guide RNA-second ribozyme fusion RNA transcribed in the cell at the 3′ end of the guide RNA, thereby forming a guide RNA that does not carry additional nucleotide at the 3′ end. In some embodiments, the 5′ end of the guide RNA coding sequence is linked to the 3′ end of a first ribozyme coding sequence, and the 3′ end of the guide RNA coding sequence is linked to the 5′ end of a second ribozyme coding sequence, wherein the first ribozyme is designed to cleave the first ribozyme-guide RNA-second ribozyme fusion RNA transcribed in the cell at the 5′ end of the guide RNA, and the second ribozyme is designed to cleave the first ribozyme-guide RNA-second ribozyme fusion RNA transcribed in the cell at the 3′ end of the guide RNA, thereby forming a guide RNA that does not carry additional nucleotides at the 5′ and 3′ ends.

The design of the first or second ribozyme is within the skill of those skilled in the art. See, for example, Gao et al., JIPB, April, 2014; Vol 56, Issue 4, 343-349.

In one specific embodiment, the first ribozyme is encoded by the following sequence:

(SEQ ID NO: 31)
5′-(N)₆CTGATGAGTCCGTGAGGACGAAACGAGTAAGCTCGTC-3′,

wherein N is independently selected from A, G, C and T, and (N)₆ represents a sequence that is reverse complementary to the first 6 nucleotides of the 5′ end of the guide RNA. In one specific embodiment, the second ribozyme is encoded by the following sequence:

(SEQ ID NO: 32)
5′-GGCCGGCATGGTCCCAGCCTCCTCGCTGGCGCCGGCTGGGCAACAT
GCTTCGGCATGGCGAATGGGAC-3′.

In some embodiments, for precise production of guide RNA within the cell, in the expression construct comprising a nucleotide sequence encoding the at least one guide RNA, the 5′ end of the guide RNA coding sequence is linked to 3′ end of a first tRNA coding sequence, wherein the first tRNA is designed to cleave the first tRNA-guide RNA fusion RNA transcribed in the cell at the 5′ end of the guide RNA (i.e., cleaved by the precise tRNA-processing machinery present within the cell that precisely excises the 5′ and 3′ extra sequences of the precursor tRNA to form the mature tRNA), thereby forming a guide RNA that does not carry additional nucleotide at the 5′ end. In one embodiment, the 3′ end of the guide RNA coding sequence is linked to the 5′ end of a second tRNA coding sequence, wherein the second tRNA is designed to cleave the guide RNA-second tRNA fusion RNA transcribed in the cell at the 3′ end of the guide RNA, thereby forming a guide RNA that does not carry additional nucleotide at the 3′ end. In some embodiments, the 5′ end of the guide RNA coding sequence is linked to the 3′ end of a first tRNA coding sequence, and the 3′ end of the guide RNA coding sequence is linked to the 5′ end of a second tRNA coding sequence, wherein the first tRNA is designed to cleave the first tRNA-guide RNA-second tRNA fusion RNA transcribed in the cell at the 5′ end of the guide RNA, and the second tRNA is designed to cleave the first tRNA-guide RNA-second tRNA fusion RNA transcribed in the cell at the 3′ end of the guide RNA, thereby forming a guide RNA that does not carry additional nucleotides at the 5′ and 3′ ends.

The design of such tRNA-guide RNA fusions is within the skill of those skilled in the art. For example, see Xie et al., PNAS, Mar. 17, 2015; vol. 112, no. 11, 3570-3575.

### 3. Method for Site-Directed Modification of Target Nucleic Acid Sequence in the Genome of a Cell

In another aspect, the present invention provides a method for site-directed modification of a target nucleic acid sequence in the genome of a cell, comprising introducing the genome editing system of the present invention into the cell.

In some embodiments, introduction of the genome editing system results in a double-strand break (DSB) in the target nucleic acid sequence. Then, substitution, deletion and/or addition of one or more nucleotides in the target nucleic acid sequence or near the target nucleic acid sequence are achieved through the repair function of the cell.

In another aspect, the present invention also provides a method of producing a genetically modified cell, comprising introducing the genome editing system of the present invention into the cell.

In another aspect, the present invention also provides a genetically modified organism comprising the genetically modified cell produced by the method of the present invention or progeny cell thereof.

In the present invention, the target sequence to be modified may be located at any location in the genome, for example, in a functional gene such as a protein-encoding gene, or may be, for example, located in a gene expression regulatory region such as a promoter region or an enhancer region, thereby the gene functional modification or gene expression modification can be achieved. Modifications in the target sequence of the cell can be detected by T7EI, PCR/RE or sequencing methods.

In the methods of the present invention, the gene editing system can be introduced into cells by a variety of methods well known to those skilled in the art.

Methods that can be used to introduce a gene editing system of the present invention into a cell include, but are not limited to, calcium phosphate transfection, protoplast fusion, electroporation, lipofection, microinjection, viral infection (e.g., baculovirus, vaccinia virus, adenovirus, adeno-associated virus, lentivirus and other viruses), gene gun method, PEG-mediated protoplast transformation, Agrobacterium-mediated transformation.

In some embodiments, the method of the invention is performed in vitro. For example, the cell is an isolated cell, or a cell in an isolated tissue or organ.

In some other embodiments, the method of the present invention can also be performed in vivo. For example, the cell is a cell within an organism, and the system of the invention can be introduced in vivo by, for example, a virus or Agrobacterium-mediated method.

The cell that can be genome-edited by the Cas12a protein or the genome editing system of the present invention is derived from an eukaryote, including but not limited to, a mammal such as human, mouse, rat, monkey, dog, pig, sheep, cattle, cat; poultry such as chicken, duck, goose; a plant including monocotyledonous and dicotyledonous plant, such as rice, corn, wheat, sorghum, barley, soybean, peanut, Arabidopsis, etc.

Preferably, the Cas12a protein or genome editing system of the present invention is particularly suitable for genome editing in plants due to being derived from plant symbiotic bacteria.

Accordingly, the present invention provides a method of producing a genetically modified plant comprising introducing the genome editing system of the present invention into at least one plant, thereby resulting in a modification in the genome of said at least one plant. The modifications include substitution, deletion and/or addition of one or more nucleotides.

In the method of the present invention, the genome editing system may be introduced into the plant with a variety of methods well known to those skilled in the art. The methods used for introducing the genome editing system of the present invention into a plant include, but are not limited to gene gun method, PEG-mediated protoplast transformation, Agrobacterium-mediated transformation, plant virus-mediated transformation, pollen tube pathway and ovary injection method.

In the method of the present invention, the modification of the target sequence can be achieved by only introducing or producing the Cas12a protein and the guide RNA in the plant cell, and the modification can be stably inherited, without any need to stably transform the genome editing system into the plant. This avoids the potential off-target effect of the stable genome editing system and also avoids the integration of the exogenous nucleotide sequence in the plant genome, thereby providing greater biosafety.

In some preferred embodiments, the introduction is carried out in the absence of selection pressure to avoid integration of the exogenous nucleotide sequence into the plant genome.

In some embodiments, the introduction comprises transforming the genome editing system of the present invention into an isolated plant cell or tissue and then regenerating the transformed plant cell or tissue into an intact plant. Preferably, the regeneration is carried out in the absence of selection pressure, i.e., no selection agent for the selection gene on the expression vector is used during tissue culture. Avoiding the use of a selection agent can increase the regeneration efficiency of the plant, obtaining a modified plant free of exogenous nucleotide sequences.

In some other embodiments, the genome editing system of the present invention can be transformed into specific parts of an intact plant, such as leaves, shoot tips, pollen tubes, young ears or hypocotyls. This is particularly suitable for the transformation of plants that are difficult to regenerate in tissue culture.

In some embodiments of the invention, the in vitro expressed protein and/or the in vitro transcribed RNA molecule are directly transformed into the plant. The protein and/or RNA molecule is capable of performing genome editing in plant cells and is subsequently degraded by the cell, avoiding integration of the exogenous nucleotide sequence in the plant genome.

Thus, in some embodiments, genetic modification of a plant using the method of the present invention may result in a plant whose genome is free of integration of exogenous polynucleotide, i.e., a transgene-free modified plant.

In some embodiments of the present invention, the modification is associated with plant traits, such as agronomic traits. For example, the modification results in a plant having altered (preferably improved) traits, such as agronomic traits, relative to a wild type plant.

In some embodiments, the method further comprises the step of screening a plant having the desired modification and/or desired traits, such as agronomic traits.

In some embodiments of the present invention, the method further comprises the step of obtaining progenies of the genetically modified plant. Preferably, the genetically modified plant or the progenies thereof have the desired modification and/or the desired traits, such as agronomic traits.

In another aspect, the invention also provides a genetically modified plant or progenies or a part thereof, wherein the plant is obtained by the method according to the present invention as described above. In some embodiments, the genetically modified plant or the progenies or a part thereof is transgene-free. Preferably, the genetically modified plant or progenies thereof have the desired genetic modification and/or the desired traits, such as agronomic traits.

In another aspect, the present invention provides a method of plant breeding comprising crossing a first genetically modified plant obtained by the above method of the present invention with a second plant not containing the modification, thereby the genetic modification is introduced into the second plant. Preferably, the first genetically modified plant has the desired traits, such as the agronomic traits.

### 4. Kit

The present invention also includes a kit for use in the method of the present invention, the kit comprising the genome editing system of the present invention, and instructions for use. The kit generally include a label indicating the intended use and/or method for use of the contents of the kit. The term label includes any written or recorded material provided on or with the kit or otherwise provided with the kit.

### EXAMPLES

### Example 1. Discovery of CRISPR/Cas12a System from Plant Symbiotic Bacteria Using Homologous Similarity Alignment

According to the reports of Bai et al. (Functional overlap of the Arabidopsis leaf and root microbiota) and Levy et al. (Genomic features of bacterial adaptation to plants) on plant symbiotic bacteria, the inventors collected the genome sequences of plant symbiotic bacteria and used CRISPRdisco software to analyze the CRISPR systems in 4269 plant symbiotic bacteria genomes, and CRISPR systems were found to be relatively abundant in plant symbiotic bacteria, but most of them are Type I CRISPR systems in Class I, and only one Cas12a protein is Type V in Class II. The Cas12a protein is derived from Flavobacterium branchiophilum, NCBI contig ID is FQ859183.1, and GeneBank protein ID is CCB70584.1, hereinafter referred to as FbCas12a.

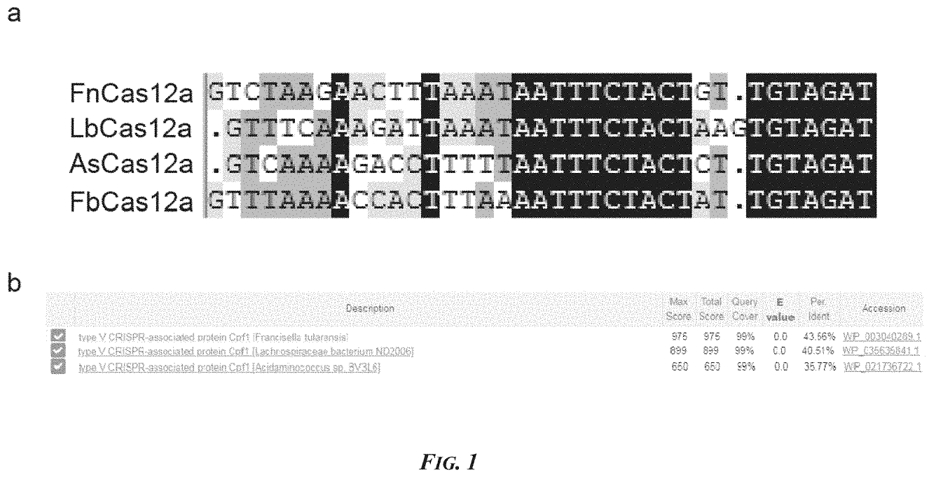

The size of the protein is 1318aa (SEQ ID NO:1), but there is no other Cas protein sequence near this sequence. The CRISPR repeat sequence begins at 1509 bp downstream in the genome. There are 37 Spacer sequences in total. The Direct Repeat is GTTTAAAACCACTTTAAAATTTCTACTATTGTAGAT (SEQ ID NO: 9). The comparison with the Direct Repeat of the commonly used Cas12a proteins FnCas12a, LbCas12a, and AsCas12a is shown in FIG. 1a. The protein sequence alignment result of this protein and commonly used FnCas12a, LbCas12a, and AsCas12a is shown in FIG. 1b, and the sequence similarity alignment was performed using the NCBI blastp program.

### Example 2. Preparation of FbCas12a and LbCas12a Expression Vectors

The vectors used for plant protoplast transformation were constructed: pJIT163-UBI-FbCas12a, pJIT163-UBI-LbCas12a, pJIT163-UBI-FbcrRNA and pJIT163-UBI-LbcrRNA.

The coding sequence of FbCas12a from Flavobacterium branchiophilum was codon-optimized, and two nuclear localization signals (NLS) were added at the 3′ end, and BamHI/SmaI restriction sites were added at the two ends, allowing better expression and localization of the FbCas12a protein in rice. The nucleotide coding sequence of FbCas12a after addition of NLSs and codon optimization is shown in SEQ ID NO: 7 in the sequence listing. In SEQ ID NO: 7, the 3967th-3987th position is the SV40 nuclear localization signal sequence, the 3988th-3999th position is the SGGS linker between the two nuclear localization signal sequences, the 4000th-4047th position is the nucleoplasmin nuclear localization signal sequence, and the 1st-3966 position is the coding sequence of FbCas12a protein. SEQ ID NO:7 encodes the protein shown in SEQ ID NO:6, i.e., the FbCas12a nuclease with nuclear localization signals.

The DNA shown in SEQ ID NO: 7 with BamHI/SmaI sites was artificially synthesized. After double digestion with BamHI/SmaI, the DNA fragment was ligated into the expression vector pJIT163 (Guerineau, F., Lucy, A. & Mullineaux, P., Effect of two consensus sequences preceding the translation initiator codon on gene expression in plant protoplasts. Plant Molecular Biology 18, 815-818, 1992)(The vector is publicly available from the Institute of Genetics and Developmental Biology, Chinese Academy of Sciences), and the resulting construct was named pJIT163-FbCas12a. It was confirmed by sequencing that a nucleotide fragment with the sequence shown in SEQ ID NO: 7 was inserted between the BamH I and SmaI restriction sites of the pJIT163 expression vector.

The DNA sequence of LbCas12a commonly used in laboratory genome editing was ligated into the pJIT163 vector to obtain the pJIT163-UBI-LbCas12a vector. The construction of the vector was similar to that of pJIT163-UBI-FbCas12a. The codon-optimized nucleotide coding sequence of LbCas12a is shown in SEQ ID NO: 8 in the sequence listing.

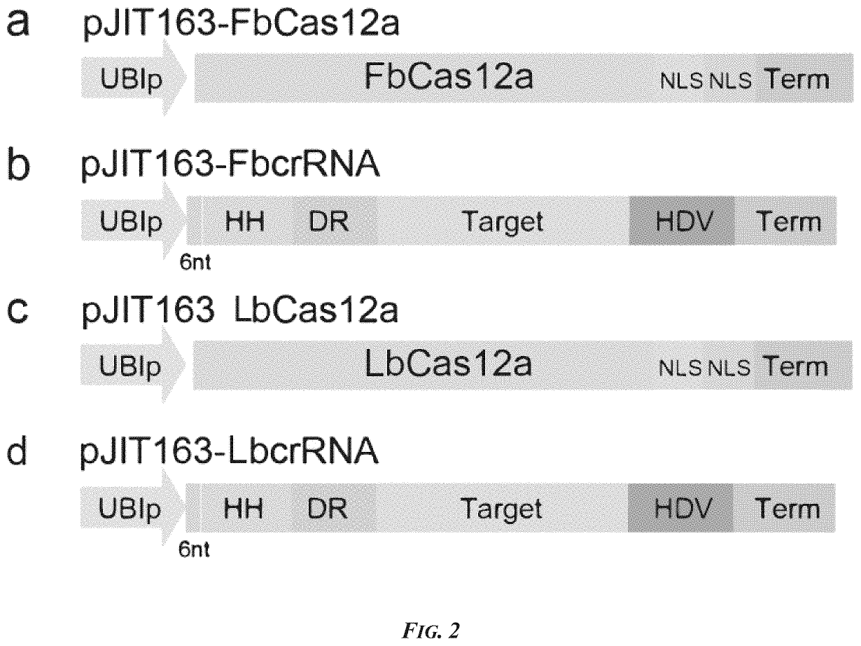

In general, pJIT163-FbCas12a and pJIT163-LbCas12a vectors contain UBI promoter, plant codon-optimized FbCas12a protein or LbCas12a coding sequence, 3′ SV40 nuclear localization signal coding sequence, nucleoplasmin nuclear localization signal coding sequence, and their structures are shown in FIG. 2a and FIG. 2c.

### Example 3. Preparation of the Vectors pJIT163-FbcrRNA with the Artificially Predicted Mature crRNA Backbone Coding Sequence of FbCas12a and the pJIT163-LbcrRNA

Cas12a has the crRNA self-mature function, however, unexpectedly, when the inventors used the full-length FbCas12a crRNA backbone sequence (full-length Direct Repeat), genome editing was not achieved. Therefore, it appears that FbCas12a does not mature its native crRNA backbone. The crRNA of FbCas12a needs to be explored to determine whether it can achieve genome editing.

The DNA fragment of the nucleotide sequence shown in SEQ ID NO: 14 was artificially synthesized, and the fragment contains hammerhead ribozyme (Hammerhead, HH ribozyme) and hepatitis delta virus ribozyme (Hepatitis deltavirus, HDV ribozyme), which can cleave the artificially predicted mature direct repeat (DR) corresponding to FbCas12a. Positions 1-6 of the fragment are the HindIII restriction site, positions 7-12 are the reverse complementary sequence required for HH ribozyme to work, positions 13-49 are the HH ribozyme sequence, positions 50-68 are artificially truncated DR sequence contains, and two BsaI restriction sites are contained at positions 69-88. The recognition sequence of the target sequence to be mutated in rice can be linked into the vector pJIT163-FbcrRNA through the two restriction sites. Positions 89-156 are HDV ribozyme sequence, and positions 157-162 is the SmaI restriction site sequence.

After HindIII/SmaI double digestion, the synthesized DNA fragment of SEQ ID NO: 14 was ligated into the expression vector pJIT163 to obtain the pJIT163-FbcrRNA vector. The vector contains UBI promoter, HH ribozyme, truncated FbcrRNA sequence, HDV ribozyme and CaMV terminator, and its structure is shown in FIG. 2b. The pJIT163-FbcrRNA vector uses a ribozyme-based crRNA maturation strategy to obtain precisely processed crRNA sequences.

The DNA fragment of the nucleotide sequence shown in SEQ ID NO: 15 (the fragment differs from FbCas12a only in the DR sequence) was artificially synthesized, double digested by HindIII/SmaI, and ligated into the expression vector pJIT163 to obtain the pJIT163-LbcrRNA vector. A schematic diagram of the structure of the vector is shown in FIG. 2d.

### Example 4. Site-Directed Mutation of Rice Endogenous Gene EPSPS by FbCas12a System and Mutation of Four Endogenous Gene Targets in Rice Using FbCas12a Protein and LbCas12a crRNA

### (1) Design of the Target Fragments:

target-EPSPS05:
TTTGGTACTAAATATACAATCCCTTGG (SEQ ID NO: 16; the
sequence is nucleotides 956-982 in the OsEPSPS
gene of LOC_Os06g04280.1; the underlined is the
PAM sequence).
target-OsCDC48:
TTTATTCAGATTACATATGGTTAG (SEQ ID NO: 17; the
sequence is nucleotides 582-605 in the OsCDC48
gene of LOC_Os03g05730; the underlined is the
PAM sequence).
target-OsDEP1T3:
TTTCAAATGGATCTAAACAGGGCCTTA (SEQ ID NO: 18; the
sequence is nucleotides 1919-1945 in the OsDEP1
gene of LOC_Os09g26999; the underlined is the
PAM sequence).
target-OsPDS:
TTTGGAGTGAAATCTCTTGTCTTA (SEQ ID NO: 19; the
sequence is nucleotides 136-159 in the OsPDS gene
of LOC_Os03g08570; the underlined is the PAM
sequence).
target-OsEpspsC02:
TTTATGAAAATATGTATGGAATTCATG (SEQ ID NO: 20; the
sequence is nucleotides 1294-1320 in the OsEPSPS
gene of LOC_Os06g04280.1; the underlined is the
PAM sequence).

(2) pJIT163-FbcrRNA Plasmid and pJIT163-LbcrRNA Plasmid Containing SP1, SP2, SP3, SP4, SP5

SP1 is the DNA encoding the RNA that can complementarily bind to the target-EPSPS05

The following single-stranded primers with sticky ends (underlined) were synthesized:

SP1-F:
(SEQ ID NO: 21)
AGATGTACTAAATATACAATCCCTTGG
SP1-R:
(SEQ ID NO: 22)
AAACCCAAGGGATTGTATATTTAGTAC

After primer annealing, a double-stranded DNA with sticky ends was formed, which was inserted between the two BsaI restriction sites of pJIT163-FbcrRNA to obtain a pJIT163-FbcrRNA plasmid containing SP1. The plasmid was confirmed as positive by sequencing.

SP2 to SP5 are DNAs encoding RNAs that can complementarily bind to target-OsCDC48, target-OsDEP1T3, target-OsPDS and target-OsEpspsC02.

The following single-stranded primers with sticky ends (underlined) were synthesized:

SP2-F:
(SEQ ID NO: 23)
AGATTTCAGATTACATATGGTTAG
SP2-R:
(SEQ ID NO: 24)
AAACCTAACCATATGTAATCTGAA
SP3-F:
(SEQ ID NO: 25)
AGATAAATGGATCTAAACAGGGCCTTA
SP3-R:
(SEQ ID NO: 26)
AAACATTGGCCCTGTTTAGATCCATTT
SP4-F:
(SEQ ID NO: 27)
AGATGAGTGAAATCTCTTGTCTTA
SP4-R:
(SEQ ID NO: 28)
AAACTAAGACAAGAGATTTCACTC
SP5-F:
(SEQ ID NO: 29)
AGATTGAAAATATGTATGGAATTCATG
SP5-R:
(SEQ ID NO: 30)
AAACCATGAATTCCATACATATTTTCA

After primer annealing, double-stranded DNAs with sticky ends were formed, which were inserted between the two BsaI restriction sites of pJIT163-FbcrRNA and pJIT163-LbcrRNA to obtain pJIT163-FbcrRNA plasmids and pJIT163-LbcrRNA plasmids containing SP1-SP5. The plasmids were confirmed to be positive by sequencing.

(3) Transformation of FbcrRNA:FbCas12a, LbcrRNA:FbCas12a, LbcrRNA:LbCas12a into Rice Protoplasts

The pJIT163-UBI-FbCas12a, pJIT163-UBI-FbCas12a, and pJIT163-FbcrRNA and pJIT163-LbcrRNA plasmids containing SP1-SP5 were respectively transformed into the protoplasts of rice Nipponbare. The specific process of rice protoplast transformation refers to Shan, Q. et al., Rapid and efficient gene modification in rice and Brachypodium using TALENs. Method disclosed in Molecular Plant (2013).

The genomic DNA was extracted 48 hours after rice protoplast transformation, and the DNA was used as a template to perform amplicon high-throughput sequencing to analyze the editing efficiency. The specific process of amplicon high-throughput sequencing refers to the Zhang et al. Perfectly matched 20-nucleotide guide RNA sequences enable robust genome editing using high-fidelity SpCas9 nucleases. Methods described in Genome Biology, 2017.

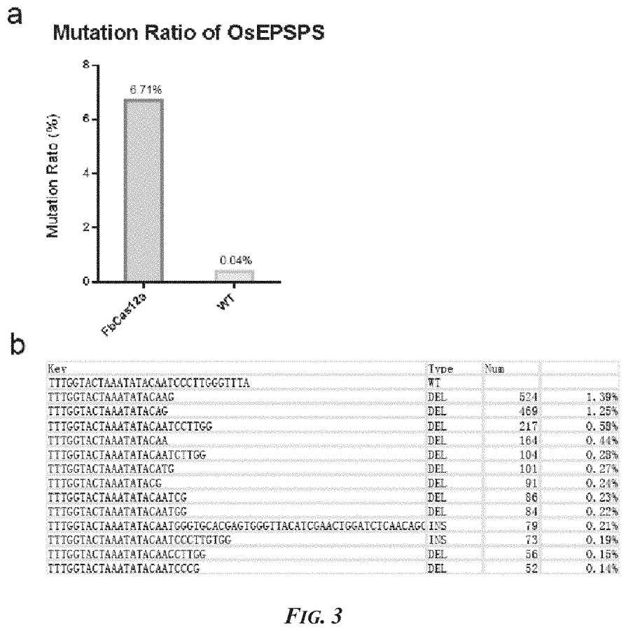

The results of the artificial matured FbcrRNA: FbCas12a high-throughput sequencing experiment are shown in FIG. 3a. The results show that, compared with the control group, the FbCas12a treatment group mutated at the target site of the OsEPSPS gene, and the mutation efficiency was about 6.25%. FIG. 3b shows the proportion of mutation types obtained from the analysis of high-throughput sequencing data. The results show that most of the mutation types generated by FbCas12a at the target site are deletions of DNA fragments.

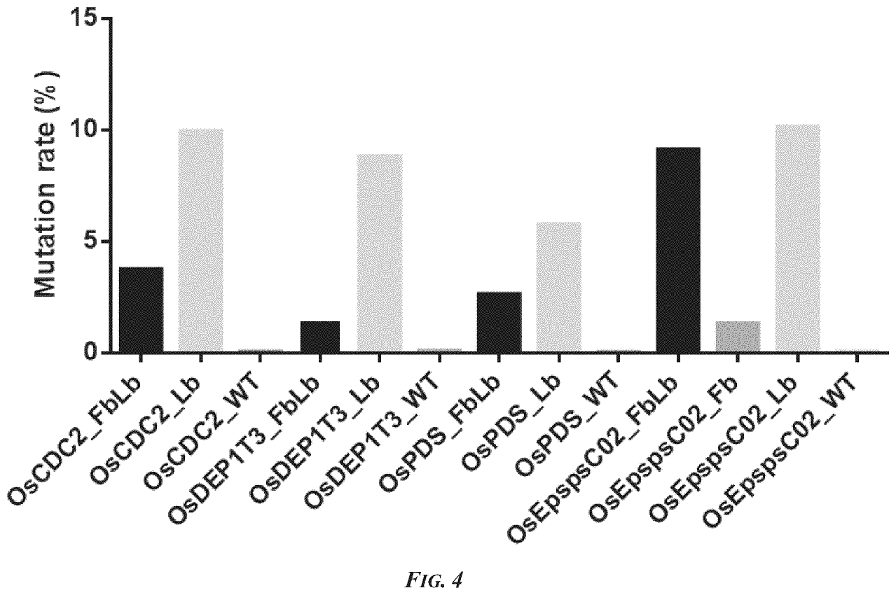

The results of another protoplast transformation experiment are shown in FIG. 4. Fb represents FbcrRNA:FbCas12a, FbLb represents LbcrRNA:FbCas12a, and Lb represents LbcrRNA:LbCas12a. The results showed that FbcrRNA:FbCas12a works at all four targets in rice, but FbCas12a works more efficiently when LbcrRNA is used.

1. A Cas12a protein which comprises
(i) an amino acid sequence having at least 80%, at least 85%, at least 90%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9%, or even 100% sequence identity to SEQ ID NO: 1, or
(ii) an amino acid sequence having substitution, deletion or addition of one or more, such as 1, 2, 3, 4, 5, 6, 7, 8, 9 or 10 amino acids as compared to SEQ ID NO: 1.

2. The Cas12a protein of claim 1, wherein the Cas12a protein is derived from a species of Flavobacterium, for example, the Cas12a protein is derived from Flavobacterium branchiophilum.

3. The Cas12a protein of claim 1, wherein the Cas12a protein further comprises a nuclear localization sequence (NLS).

4. The Cas12a protein of claim 3, which comprises the amino acid sequence shown in SEQ ID NO:6.

5. Use of the Cas12a protein of claim 1 for genome editing in a cell, preferably an eukaryotic cell, more preferably a plant cell.

6. A genome editing system for site-directed modification of a target nucleic acid sequence in the genome of a cell, comprising the Cas12a protein of claim 1 and/or an expression construct comprising a nucleotide sequence encoding the Cas12a protein of claim 1.

7. The genome editing system of claim 6, further comprises at least one guide RNA (gRNA) and/or an expression construct comprising a nucleotide sequence encoding the at least one guide RNA.

8. The genome editing system of claim 7, wherein the guide RNA is a crRNA, and comprises the crRNA scaffold sequence set forth in SEQ ID NO: 10 or 11.

9. The genome editing system of claim 7, wherein the genome editing system comprises:
i) the Cas12a protein of claim 1 and the at least one guide RNA, optionally, the Cas12a protein and the at least one guide RNA form a complex;
ii) an expression construct comprising a nucleotide sequence encoding the Cas12a protein of claim 1, and the at least one guide RNA;
iii) a Cas12a protein of claim 1, and an expression construct comprising a nucleotide sequence encoding the at least one guide RNA;
iv) an expression construct comprising a nucleotide sequence encoding the Cas12a protein of claim 1, and an expression construct comprising a nucleotide sequence encoding the at least one guide RNA; or
v) an expression construct comprising a nucleotide sequence encoding the Cas12a protein of claim 1 and a nucleotide sequence encoding the at least one guide RNA.

10. The genome editing system of claim 6, wherein the nucleotide sequence encoding the Cas12a protein is codon-optimized for plants such as rice.

11. The genome editing system of claim 10, wherein the nucleotide sequence encoding the Cas12a protein is selected from the group consisting of SEQ ID NO:2 and SEQ ID NO:7.

12. The genome editing system of claim 7, wherein the nucleotide sequence encoding the Cas12a protein and/or the nucleotide sequence encoding the at least one guide RNA is operably linked to an expression regulatory element, such as a promoter.

13. The genome editing system of claim 7, wherein the 5′ end of the guide RNA coding sequence is linked to the 3′ end of a first ribozyme coding sequence, and the 3′ end of the guide RNA coding sequence is linked to the 5′ end of a second ribozyme coding sequence, wherein the first ribozyme is designed to cleave the first ribozyme-guide RNA-second ribozyme fusion RNA transcribed in the cell at the 5′ end of the guide RNA, and the second ribozyme is designed to cleave the first ribozyme-guide RNA-second ribozyme fusion RNA transcribed in the cell at the 3′ end of the guide RNA, thereby forming a guide RNA that does not carry additional nucleotides at the 5′ and 3′ ends.

14. The genome editing system of claim 13, wherein the first ribozyme is encoded by the sequence shown in SEQ ID NO:31, and the second ribozyme is encoded by the sequence shown in SEQ ID NO:32.

15. A method of producing a genetically modified cell, comprising introducing the genome editing system of claim 6 into the cell.

16. The method of claim 15, wherein the cell is derived from a mammal such as human, mouse, rat, monkey, dog, pig, sheep, cattle, cat; poultry such as chicken, duck, goose; a plant including monocotyledonous and dicotyledonous plant, such as rice, corn, wheat, sorghum, barley, soybean, peanut, Arabidopsis.


SEQUENCE LISTING (32 sequences)

SEQ ID NO: 1 - protein, 1318 aa, Flavobacterium branchiophilum
MTNKFTNQYSLSKTLRFELIPQGKTLEFIQEKGLLSQDKQRAESYQEMKKTIDKFHKYFIDLALSNAKLTHLETYLELYNKSAETKKEQKFKDDLKKVQDNLRKEIVKSFSDGDAKSIFAILDKKELITVELEKWFENNEQKDIYFDEKFKTFTTYFTGFHQNRKNMYSVEPNSTAIAYRLIHENLPKFLENAKAFEKIKQVESLQVNFRELMGEFGDEGLIFVNELEEMFQINYYNDVLSQNGITIYNSIISGFTKNDIKYKGLNEYINNYNQTKDKKDRLPKLKQLYKQILSDRISLSFLPDAFTDGKQVLKAIFDFYKINLLSYTIEGQEESQNLLLLIRQTIENLSSFDTQKIYLKNDTHLTTISQQVFGDFSVFSTALNYWYETKVNPKFETEYSKANEKKREILDKAKAVFTKQDYFSIAFLQEVLSEYILTLDHTSDIVKKHSSNCIADYFKNHFVAKKENETDKTFDFIANITAKYQCIQGILENADQYEDELKQDQKLIDNLKFFLDAILELLHFIKPLHLKSESITEKDTAFYDVFENYYEALSLLTPLYNMVRNYVTQKPYSTEKIKLNFENAQLLNGWDANKEGDYLTTILKKDGNYFLAIMDKKHNKAFQKFPEGKENYEKMVYKLLPGVNKMLPKVFFSNKNIAYFNPSKELLENYKKETHKKGDTFNLEHCHTLIDFFKDSLNKHEDWKYFDFQFSETKSYQDLSGFYREVEHQGYKINFKNIDSEYIDGLVNEGKLFLFQIYSKDFSPFSKGKPNMHTLYWKALFEEQNLQNVIYKLNGQAEIFFRKASIKPKNIILHKKKIKIAKKHFIDKKTKTSEIVPVQTIKNLNMYYQGKISEKELTQDDLRYIDNFSIFNEKNKTIDIIKDKRFTVDKFQFHVPITMNFKATGGSYINQTVLEYLQNNPEVKIIGLDRGERHLVYLTLIDQQGNILKQESLNTITDSKISTPYHKLLDNKENERDLARKNWGTVENIKELKEGYISQVVHKIATLMLEENAIVVMEDLNFGFKRGRFKVEKQIYQKLEKMLIDKLNYLVLKDKQPQELGGLYNALQLTNKFESFQKMGKQSGFLFYVPAWNTSKIDPTTGFVNYFYTKYENVDKAKAFFEKFEAIRFNAEKKYFEFEVKKYSDFNPKAEGTQQAWTICTYGERIETKRQKDQNNKFVSTPINLTEKIEDFLGKNQIVYGDGNCIKSQIASKDDKAFFETLLYWFKMTLQMRNSETRTDIDYLISPVMNDNGTFYNSRDYEKLENPTLPKDADANGAYHIAKKGLMLLNKIDQADLTKKVDLSISNRDWLQFVQKNK

SEQ ID NO: 2 - DNA, 3954 bp, Flavobacterium branchiophilum
atgaccaacaagttcacaaatcagtactccctgagcaagaccctccgcttcgagctgattccgcagggcaagacactcgagttcatccaggagaagggcctcctgtcccaggacaagcagagggccgagagctaccaggagatgaagaagaccattgacaagttccacaagtacttcatcgatctcgccctgtcaaatgcgaagctcacccatctggagacatacctcgagctgtacaacaagtctgcggagacaaagaaggagcagaagttcaaggacgatctcaagaaggtccaggacaacctgaggaaggagatcgtgaagtcattctctgacggcgatgccaagagcatcttcgcgattctggataagaaggagctgatcaccgtggagctggagaagtggttcgagaacaatgagcagaaggacatctacttcgatgagaagttcaagaccttcaccacatacttcacaggcttccaccagaacaggaagaatatgtactcagtcgagccaaactctacagccatcgcgtaccgcctcattcatgagaacctccctaagttcctggagaatgccaaggcgttcgagaagatcaagcaggtggagtccctccaggtcaatttccgcgagctgatgggcgagttcggcgacgagggcctcatcttcgtcaacgagctggaggagatgttccagattaactactacaatgatgtgctcagccagaacggcatcaccatctacaattcaatcatttctggcttcacaaagaacgacattaagtacaagggcctgaacgagtacatcaacaattacaatcagaccaaggacaagaaggatcgcctcccgaagctgaagcagctctacaagcagatcctctccgaccggatttccctgagcttcctcccagacgccttcaccgatggcaagcaggtgctgaaggcgattttcgatttctacaagatcaatctcctgtcctacacaatcgagggccaggaggagagccagaatctcctgctcctgatccggcagacaattgagaacctctccagcttcgacacccagaagatctacctcaagaacgatacacacctgaccacaatttcccagcaggtgttcggcgatttctccgtcttcagcaccgccctcaactactggtacgagacaaaagtgaatccgaagttcgagacagagtacagcaaggcgaacgagaagaagagggagatcctggacaaggccaaggcggtcttcaccaagcaggattacttctcaattgccttcctccaggaggtgctgtctgagtacattctcaccctggaccacacatccgatatcgtcaagaagcattcatctaactgcattgccgactacttcaagaatcatttcgtcgcgaagaaggagaacgagacagacaagacattcgatttcatcgccaatattacagcgaagtaccagtgcatccagggcattctcgagaacgccgaccagtacgaggatgagctgaagcaggaccagaagctcattgataatctgaagttcttcctcgatgcgatcctcgagctgctgcacttcatcaagccactccatctgaagtcagagtctatcaccgagaaggacacagccttctacgatgtgttcgagaactactacgaggcgctctccctcctgacccctctgtacaacatggtgaggaattacgtcacccagaagccgtacagcacagagaagatcaagctcaacttcgagaatgcccagctcctgaacggctgggacgcgaataaggagggcgattacctcaccacaatcctgaagaaggacggcaattacttcctcgccattatggataagaagcacaacaaggcgttccagaagttccctgagggcaaggagaattacgagaagatggtgtacaagctcctgccaggcgtcaacaagatgctccctaaggtgttcttctccaacaagaatatcgcctacttcaatccgagcaaggagctgctcgagaactacaagaaggagacacacaagaagggcgacacattcaatctcgagcactgccataccctgatcgacttcttcaaggattccctcaacaagcatgaggactggaagtacttcgatttccagttctcagagacaaagtcttaccaggatctgagcggcttctaccgcgaggtggagcaccagggctacaagatcaacttcaagaatattgactcagagtacatcgatggcctcgtcaatgagggcaagctcttcctgttccagatctactctaaggacttctcacctttctctaagggcaagccgaacatgcataccctctactggaaggccctgttcgaggagcagaacctccagaatgtgatctacaagctgaatggccaggccgagattttcttccggaaggcgtcaatcaagccaaagaacatcattctccacaagaagaagatcaagattgcgaagaagcatttcatcgataagaagaccaagacatctgagattgtgcctgtccagaccatcaagaacctcaatatgtactaccagggcaagatttccgagaaggagctgacacaggacgatctgcggtacattgacaacttcagcatcttcaacgagaagaataagaccatcgacatcattaaggataagaggttcacagtcgataagttccagttccacgtgcctatcaccatgaatttcaaggccacaggcggctcatacattaaccagaccgtcctggagtacctccagaacaatccggaggtgaagatcattggcctcgaccggggcgagaggcacctggtgtacctcaccctgattgatcagcagggcaatatcctcaagcaggagtccctgaacaccattacagactccaagatcagcacaccataccataagctcctggacaacaaggagaatgagcgcgatctcgcgcggaagaactggggcaccgtcgagaatatcaaggagctgaaggagggctacatttcccaggtggtccataagatcgccacactcatgctggaggagaatgcgattgtggtcatggaggatctcaacttcggcttcaagaggggccgcttcaaggtggagaagcagatctaccagaagctcgagaagatgctgattgacaagctcaattacctcgtcctgaaggataagcagcctcaggagctgggcggcctctacaacgccctccagctgaccaataagttcgagtcattccagaagatgggcaagcagtctggcttcctcttctacgtgccggcgtggaacaccagcaagattgacccaaccacaggcttcgtcaattacttctacacaaagtacgagaacgtggataaggccaaggcgttcttcgagaagttcgaggccatccgcttcaatgcggagaagaagtacttcgagttcgaggtcaagaagtactcagacttcaatccaaaggcggagggaacccagcaggcgtggaccatttgcacatacggcgagaggatcgagacaaagcgccagaaggatcagaacaataagttcgtgtctaccccaattaatctcacagagaagatcgaggacttcctgggcaagaatcagatcgtctacggcgatggcaactgcatcaagtcccagattgccagcaaggacgataaggcgttcttcgagacactcctgtactggttcaagatgaccctccagatgcggaactcagagacaaggacagacatcgattacctgatttctccagtgatgaacgacaatggcaccttctacaattccagggattacgagaagctcgagaacccgacactgccaaaggacgccgatgcgaatggcgcctaccatatcgcgaagaagggcctcatgctcctgaacaagattgaccaggccgatctcaccaagaaggtcgacctgtccatcagcaatcgcgattggctgcagttcgtgcagaagaacaag

SEQ ID NO: 3 - protein, 4 aa, Artificial Sequence
  Artificially synthesized peptide linker
SGGS

SEQ ID NO: 4 - protein, 7 aa, Artificial Sequence
  Artificially synthesized SV40 nuclear localization sequence
PKKKRKV

SEQ ID NO: 5 - protein, 16 aa, Artificial Sequence
  Artificially synthesized nucleoplasmin nuclear localization sequence
KRPAATKKAGQAKKKK

SEQ ID NO: 6 - protein, 1349 aa, Artificial Sequence
  Artificially synthesized FbCas12a amino acid sequence with nuclear localization sequence
MTNKFTNQYSLSKTLRFELIPQGKTLEFIQEKGLLSQDKQRAESYQEMKKTIDKFHKYFIDLALSNAKLTHLETYLELYNKSAETKKEQKFKDDLKKVQDNLRKEIVKSFSDGDAKSIFAILDKKELITVELEKWFENNEQKDIYFDEKFKTFTTYFTGFHQNRKNMYSVEPNSTAIAYRLIHENLPKFLENAKAFEKIKQVESLQVNFRELMGEFGDEGLIFVNELEEMFQINYYNDVLSQNGITIYNSIISGFTKNDIKYKGLNEYINNYNQTKDKKDRLPKLKQLYKQILSDRISLSFLPDAFTDGKQVLKAIFDFYKINLLSYTIEGQEESQNLLLLIRQTIENLSSFDTQKIYLKNDTHLTTISQQVFGDFSVFSTALNYWYETKVNPKFETEYSKANEKKREILDKAKAVFTKQDYFSIAFLQEVLSEYILTLDHTSDIVKKHSSNCIADYFKNHFVAKKENETDKTFDFIANITAKYQCIQGILENADQYEDELKQDQKLIDNLKFFLDAILELLHFIKPLHLKSESITEKDTAFYDVFENYYEALSLLTPLYNMVRNYVTQKPYSTEKIKLNFENAQLLNGWDANKEGDYLTTILKKDGNYFLAIMDKKHNKAFQKFPEGKENYEKMVYKLLPGVNKMLPKVFFSNKNIAYFNPSKELLENYKKETHKKGDTFNLEHCHTLIDFFKDSLNKHEDWKYFDFQFSETKSYQDLSGFYREVEHQGYKINFKNIDSEYIDGLVNEGKLFLFQIYSKDFSPFSKGKPNMHTLYWKALFEEQNLQNVIYKLNGQAEIFFRKASIKPKNIILHKKKIKIAKKHFIDKKTKTSEIVPVQTIKNLNMYYQGKISEKELTQDDLRYIDNFSIFNEKNKTIDIIKDKRFTVDKFQFHVPITMNFKATGGSYINQTVLEYLQNNPEVKIIGLDRGERHLVYLTLIDQQGNILKQESLNTITDSKISTPYHKLLDNKENERDLARKNWGTVENIKELKEGYISQVVHKIATLMLEENAIVVMEDLNFGFKRGRFKVEKQIYQKLEKMLIDKLNYLVLKDKQPQELGGLYNALQLTNKFESFQKMGKQSGFLFYVPAWNTSKIDPTTGFVNYFYTKYENVDKAKAFFEKFEAIRFNAEKKYFEFEVKKYSDFNPKAEGTQQAWTICTYGERIETKRQKDQNNKFVSTPINLTEKIEDFLGKNQIVYGDGNCIKSQIASKDDKAFFETLLYWFKMTLQMRNSETRTDIDYLISPVMNDNGTFYNSRDYEKLENPTLPKDADANGAYHIAKKGLMLLNKIDQADLTKKVDLSISNRDWLQFVQKNKSGGSPKKKRKVSGGSKRPAATKKAGQAKKKK

SEQ ID NO: 7 - DNA, 4047 bp, Artificial Sequence
  Artificially synthesized coding sequence of FbCas12a with nuclear localization sequence
atgaccaacaagttcacaaatcagtactccctgagcaagaccctccgcttcgagctgattccgcagggcaagacactcgagttcatccaggagaagggcctcctgtcccaggacaagcagagggccgagagctaccaggagatgaagaagaccattgacaagttccacaagtacttcatcgatctcgccctgtcaaatgcgaagctcacccatctggagacatacctcgagctgtacaacaagtctgcggagacaaagaaggagcagaagttcaaggacgatctcaagaaggtccaggacaacctgaggaaggagatcgtgaagtcattctctgacggcgatgccaagagcatcttcgcgattctggataagaaggagctgatcaccgtggagctggagaagtggttcgagaacaatgagcagaaggacatctacttcgatgagaagttcaagaccttcaccacatacttcacaggcttccaccagaacaggaagaatatgtactcagtcgagccaaactctacagccatcgcgtaccgcctcattcatgagaacctccctaagttcctggagaatgccaaggcgttcgagaagatcaagcaggtggagtccctccaggtcaatttccgcgagctgatgggcgagttcggcgacgagggcctcatcttcgtcaacgagctggaggagatgttccagattaactactacaatgatgtgctcagccagaacggcatcaccatctacaattcaatcatttctggcttcacaaagaacgacattaagtacaagggcctgaacgagtacatcaacaattacaatcagaccaaggacaagaaggatcgcctcccgaagctgaagcagctctacaagcagatcctctccgaccggatttccctgagcttcctcccagacgccttcaccgatggcaagcaggtgctgaaggcgattttcgatttctacaagatcaatctcctgtcctacacaatcgagggccaggaggagagccagaatctcctgctcctgatccggcagacaattgagaacctctccagcttcgacacccagaagatctacctcaagaacgatacacacctgaccacaatttcccagcaggtgttcggcgatttctccgtcttcagcaccgccctcaactactggtacgagacaaaagtgaatccgaagttcgagacagagtacagcaaggcgaacgagaagaagagggagatcctggacaaggccaaggcggtcttcaccaagcaggattacttctcaattgccttcctccaggaggtgctgtctgagtacattctcaccctggaccacacatccgatatcgtcaagaagcattcatctaactgcattgccgactacttcaagaatcatttcgtcgcgaagaaggagaacgagacagacaagacattcgatttcatcgccaatattacagcgaagtaccagtgcatccagggcattctcgagaacgccgaccagtacgaggatgagctgaagcaggaccagaagctcattgataatctgaagttcttcctcgatgcgatcctcgagctgctgcacttcatcaagccactccatctgaagtcagagtctatcaccgagaaggacacagccttctacgatgtgttcgagaactactacgaggcgctctccctcctgacccctctgtacaacatggtgaggaattacgtcacccagaagccgtacagcacagagaagatcaagctcaacttcgagaatgcccagctcctgaacggctgggacgcgaataaggagggcgattacctcaccacaatcctgaagaaggacggcaattacttcctcgccattatggataagaagcacaacaaggcgttccagaagttccctgagggcaaggagaattacgagaagatggtgtacaagctcctgccaggcgtcaacaagatgctccctaaggtgttcttctccaacaagaatatcgcctacttcaatccgagcaaggagctgctcgagaactacaagaaggagacacacaagaagggcgacacattcaatctcgagcactgccataccctgatcgacttcttcaaggattccctcaacaagcatgaggactggaagtacttcgatttccagttctcagagacaaagtcttaccaggatctgagcggcttctaccgcgaggtggagcaccagggctacaagatcaacttcaagaatattgactcagagtacatcgatggcctcgtcaatgagggcaagctcttcctgttccagatctactctaaggacttctcacctttctctaagggcaagccgaacatgcataccctctactggaaggccctgttcgaggagcagaacctccagaatgtgatctacaagctgaatggccaggccgagattttcttccggaaggcgtcaatcaagccaaagaacatcattctccacaagaagaagatcaagattgcgaagaagcatttcatcgataagaagaccaagacatctgagattgtgcctgtccagaccatcaagaacctcaatatgtactaccagggcaagatttccgagaaggagctgacacaggacgatctgcggtacattgacaacttcagcatcttcaacgagaagaataagaccatcgacatcattaaggataagaggttcacagtcgataagttccagttccacgtgcctatcaccatgaatttcaaggccacaggcggctcatacattaaccagaccgtcctggagtacctccagaacaatccggaggtgaagatcattggcctcgaccggggcgagaggcacctggtgtacctcaccctgattgatcagcagggcaatatcctcaagcaggagtccctgaacaccattacagactccaagatcagcacaccataccataagctcctggacaacaaggagaatgagcgcgatctcgcgcggaagaactggggcaccgtcgagaatatcaaggagctgaaggagggctacatttcccaggtggtccataagatcgccacactcatgctggaggagaatgcgattgtggtcatggaggatctcaacttcggcttcaagaggggccgcttcaaggtggagaagcagatctaccagaagctcgagaagatgctgattgacaagctcaattacctcgtcctgaaggataagcagcctcaggagctgggcggcctctacaacgccctccagctgaccaataagttcgagtcattccagaagatgggcaagcagtctggcttcctcttctacgtgccggcgtggaacaccagcaagattgacccaaccacaggcttcgtcaattacttctacacaaagtacgagaacgtggataaggccaaggcgttcttcgagaagttcgaggccatccgcttcaatgcggagaagaagtacttcgagttcgaggtcaagaagtactcagacttcaatccaaaggcggagggaacccagcaggcgtggaccatttgcacatacggcgagaggatcgagacaaagcgccagaaggatcagaacaataagttcgtgtctaccccaattaatctcacagagaagatcgaggacttcctgggcaagaatcagatcgtctacggcgatggcaactgcatcaagtcccagattgccagcaaggacgataaggcgttcttcgagacactcctgtactggttcaagatgaccctccagatgcggaactcagagacaaggacagacatcgattacctgatttctccagtgatgaacgacaatggcaccttctacaattccagggattacgagaagctcgagaacccgacactgccaaaggacgccgatgcgaatggcgcctaccatatcgcgaagaagggcctcatgctcctgaacaagattgaccaggccgatctcaccaagaaggtcgacctgtccatcagcaatcgcgattggctgcagttcgtgcagaagaacaagtccggcggcagcccaaagaagaagaggaaggtgagcggcggcagcaagcggccagcggcgacgaagaaggcggggcaggcgaagaagaagaag

SEQ ID NO: 8 - DNA, 3687 bp, Artificial Sequence
  Artificially synthesized codon-optimized LbCas12a coding sequence
atgtcaaagctcgagaaattcaccaactgttattcgttgagcaaaacactgcggtttaaagcgattccagtcggcaagactcaagagaatatagacaataagcggctgttggtggaagatgaaaagcgcgcggaagactacaaaggggtgaagaagttgttggacagatactacctctcttttatcaatgatgtcttgcactcaatcaaattgaagaatctgaacaactacatctccctcttcagaaagaaaacaaggacagaaaaggagaataaggaacttgaaaatttggagatcaatctgaggaaagagatcgcgaaagcctttaaaggcaacgaaggatacaaaagtctgttcaagaaggatataattgagacaattttgccagagttcctcgatgacaaggacgagattgcgctggtcaattcgttcaacggattcacaacagcattcacaggcttctttgataatcgggaaaatatgttctctgaggaggcaaagtccacttctattgcgttcaggtgtatcaatgagaatctcactaggtacatttccaacatggatatctttgagaaggttgacgcaatttttgacaagcacgaagttcaggagattaaggagaagatcctcaattccgattatgacgttgaggacttcttcgaaggtgagttttttaatttcgtgctcactcaagagggtatcgacgtgtataatgcgatcatcggtgggttcgtgactgagtccggtgaaaagattaagggattgaacgagtatatcaacctttacaaccaaaagacgaaacagaagctgccaaagttcaagcctctttacaaacaggttctttcagaccgcgagtcactctcgttctatggggagggctacacttcggatgaggaagtcctggaggtgttcaggaatactctcaataagaattcggagattttctcttctataaaaaaactggaaaagttgtttaagaattttgacgaatactctagcgccggcatatttgtgaaaaacggcccggccatatcaacgataagtaaagatatcttcggcgaatggaacgtgatcagagacaaatggaacgcggagtatgacgatattcacctgaagaagaaggctgtcgtaacggagaagtacgaggatgatcgcaggaaaagcttcaaaaagatcggaagtttcagcctggaacagttgcaggagtatgctgacgccgatcttagcgtcgtcgagaagttgaaggagataatcatccaaaaggtcgacgagatatataaagtctatggatcaagtgaaaaactgttcgacgccgacttcgttttggagaagtccctgaagaagaacgacgctgttgttgccattatgaaggatctgctcgacagcgtgaagagtttcgagaactatattaaggcttttttcggggaggggaaggagactaacagagatgagtccttctacggagacttcgtcctcgcgtacgatatactccttaaggtagaccacatctacgacgcaatcagaaattacgtgacacaaaagccgtacagcaaggacaagttcaaactctacttccagaacccccagttcatgggcggctgggacaaggacaaggaaacggattacagggctacgatcctgaggtatggttcaaaatactacttggcgattatggacaagaagtacgccaagtgtctccagaagattgacaaagacgatgtcaatggcaattatgagaagatcaactacaagctgcttccgggtccgaacaagatgctcccaaaggttttcttcagcaagaaatggatggcctactataacccaagcgaggacatccagaagatttataagaacggtacgttcaagaagggcgacatgttcaatcttaacgactgtcacaagctgatcgacttcttcaaagactcaattagccggtacccaaagtggtctaacgcctatgacttcaacttttcggaaaccgagaagtacaaggatatagccggattttatagagaggtggaagagcagggctacaaggtgtcattcgagtccgccagcaagaaggaagtggacaagctcgtggaagagggtaagctctacatgttccagatttataataaagactttagcgataagagccacgggacacctaatctccacacaatgtatttcaagctgctcttcgacgagaataaccacggccaaatcaggttgtcaggaggggctgaactcttcatgcggcgcgctagccttaagaaggaggagcttgtagtccaccctgcgaatagtccaattgcgaataagaacccggacaatcctaaaaagactacaacattgagctacgacgtgtacaaggataagaggttttccgaggatcagtacgagctccacatcccgattgcgatcaacaagtgcccaaagaatattttcaagataaacacagaggtgcgtgtactcctgaagcatgacgacaatccttacgtcattgggattgatcggggcgagaggaacctcctctatattgtggtggtggacgggaaggggaacatagtcgaacagtactcccttaacgaaataattaacaatttcaacggcatccgtatcaagaccgactaccattcgttgctggacaagaaggagaaggagagatttgaggcgcggcaaaattggacaagtatcgagaacatcaaggaactcaaagcaggttatatctctcaagttgtgcataagatatgcgagctggttgagaagtatgacgcagtgatcgctcttgaggacctcaactcgggctttaagaattctagagttaaagtggagaagcaggtctatcaaaagttcgagaagatgcttatagataagctcaactacatggtcgataagaaatcgaacccatgtgccaccggcggcgcactcaaaggttaccaaataacaaacaaattcgagtccttcaaatcgatgagtactcagaatgggttcatattttatataccggcgtggcttacgtctaagatcgacccgtcaactggttttgtcaacctgttgaagacgaaatacacgtccattgccgattcgaaaaagttcatatctagttttgatcgtattatgtacgtcccagaggaagatcttttcgagtttgctctcgactacaaaaacttttcgcggaccgatgcggattacattaaaaaatggaaactctattcgtacggcaacagaatcaggatttttcgcaaccctaagaagaataacgtctttgattgggaggaagtttgcttgactagcgcgtacaaggagctctttaataagtatggcattaactaccaacagggtgatatcagagcactgctttgcgaacaatctgacaaggctttctactcatccttcatggctttgatgagcctgatgctccagatgagaaattcaattacaggcagaaccgacgtggatttcttgatctccccggttaaaaattctgatggcatcttttacgatagcaggaactatgaagcgcaagagaatgcgattctgccaaaaaatgcagacgccaacggtgcctataacatcgccaggaaagtcctgtgggcgatcggccagttcaaaaaggccgaagacgaaaaattggacaaggtcaaaatcgctatcagcaacaaagagtggctggagtatgctcagacatccgtaaagcattag

SEQ ID NO: 9 - DNA, 36 bp, Artificial Sequence
  Artificially synthesized DR sequence of FbCas12a
gtttaaaaccactttaaaatttctactattgtagat

SEQ ID NO: 10 - RNA, 19 nt, Artificial Sequence
  Artificially synthesized predicted crRNA scaffold sequence of FbCas12a
aauuucuacuauuguagau

SEQ ID NO: 11 - RNA, 21 nt, Artificial Sequence
  Artificially synthesized crRNA scaffold sequence of LbCas12a
uaauuucuacuaaguguagau

SEQ ID NO: 12 - DNA, 19 bp, Artificial Sequence
  Artificially synthesized coding sequence of predicted crRNA scaffold sequence of FbCas12a
aatttctactattgtagat

SEQ ID NO: 13 - DNA, 21 bp, Artificial Sequence
  Artificially synthesized coding sequence of crRNA scaffold sequence of LbCas12a
taatttctactaagtgtagat

SEQ ID NO: 14 - DNA, 162 bp, Artificial Sequence
  Artificially synthesized expression cassette of FbCas12a crRNA in combination with ribozyme
aagcttaaattactgatgagtccgtgaggacgaaacgagtaagctcgtcaatttctactattgtagattgagaccctgcagggtctctggccggcatggtcccagcctcctcgctggcgccggctgggcaacatgcttcggcatggcgaatgggaccccggg

SEQ ID NO: 15 - DNA, 164 bp, Artificial Sequence
  Artificially synthesized expression cassette of LbCas12a crRNA in combination with ribozyme
aagcttaaattactgatgagtccgtgaggacgaaacgagtaagctcgtctaatttctactaagtgtagattgagaccctgcagggtctctggccggcatggtcccagcctcctcgctggcgccggctgggcaacatgcttcggcatggcgaatgggaccccggg

SEQ ID NO: 16 - DNA, 27 bp, Artificial Sequence
  Artificially synthesized target-EPSPS05
tttggtactaaatatacaatcccttgg

SEQ ID NO: 17 - DNA, 24 bp, Artificial Sequence
  Artificially synthesized target-OsCDC48
tttattcagattacatatggttag

SEQ ID NO: 18 - DNA, 27 bp, Artificial Sequence
  Artificially synthesized target-OsDEP1T3
tttcaaatggatctaaacagggcctta

SEQ ID NO: 19 - DNA, 24 bp, Artificial Sequence
  Artificially synthesized target-OsPDS
tttggagtgaaatctcttgtctta

SEQ ID NO: 20 - DNA, 27 bp, Artificial Sequence
  Artificially synthesized target-OsEpspsC02
tttatgaaaatatgtatggaattcatg

SEQ ID NO: 21 - DNA, 27 bp, Artificial Sequence
  Artificially synthesized oligonucleotide SP1-F
agatgtactaaatatacaatcccttgg

SEQ ID NO: 22 - DNA, 27 bp, Artificial Sequence
  Artificially synthesized oligonucleotide SP1-R
aaacccaagggattgtatatttagtac

SEQ ID NO: 23 - DNA, 24 bp, Artificial Sequence
  Artificially synthesized oligonucleotide SP2-F
agatttcagattacatatggttag

SEQ ID NO: 24 - DNA, 24 bp, Artificial Sequence
  Artificially synthesized oligonucleotide SP2-R
aaacctaaccatatgtaatctgaa

SEQ ID NO: 25 - DNA, 27 bp, Artificial Sequence
  Artificially synthesized oligonucleotide SP3-F
agataaatggatctaaacagggcctta

SEQ ID NO: 26 - DNA, 27 bp, Artificial Sequence
  Artificially synthesized oligonucleotide SP3-R
aaacattggccctgtttagatccattt

SEQ ID NO: 27 - DNA, 24 bp, Artificial Sequence
  Artificially synthesized oligonucleotide SP4-F
agatgagtgaaatctcttgtctta

SEQ ID NO: 28 - DNA, 24 bp, Artificial Sequence
  Artificially synthesized oligonucleotide SP4-R
aaactaagacaagagatttcactc

SEQ ID NO: 29 - DNA, 27 bp, Artificial Sequence
  Artificially synthesized oligonucleotide SP5-F
agattgaaaatatgtatggaattcatg

SEQ ID NO: 30 - DNA, 27 bp, Artificial Sequence
  Artificially synthesized oligonucleotide SP5-R
aaaccatgaattccatacatattttca

SEQ ID NO: 31 - DNA, 43 bp, Artificial Sequence
  Artificially synthesized 5′ ribozyme sequence
  misc_feature (1)..(6): n is a, c, g, or t
nnnnnnctgatgagtccgtgaggacgaaacgagtaagctcgtc

SEQ ID NO: 32 - DNA, 68 bp, Artificial Sequence
  Artificially synthesized 3′ ribozyme sequence
ggccggcatggtcccagcctcctcgctggcgccggctgggcaacatgcttcggcatggcgaatgggac